(19) United States
(12) Patent Application Publication
(10) Pub. No.: US 2018/0070873 A1
(43) Pub. Date: Mar. 15, 2018

(54) METHODS AND SOFTWARE FOR PROVIDING HEALTH INFORMATION TO A USER EXPRESSING SYMPTOMS OF AN ALLERGIC REACTION VIA A WEARABLE DEVICE

(71) Applicant: KONINKLIJKE PHILIPS N.V., EINDHOVEN (NL)

(72) Inventors: JOHN CRONIN, BONITA SPRINGS, FL (US); CHRISTOPHER HUFFINES, WILLISTON, VT (US)

(21) Appl. No.: 15/556,316

(22) PCT Filed: Mar. 7, 2016
(86) PCT No.: PCT/EP2016/054734

§ 371 Date: Sep. 7, 2017

Related U.S. Application Data
(60) Provisional application No. 62/130,218, filed on Mar. 9, 2015.

(30) Foreign Application Priority Data
Jul. 16, 2015 (EP) 15177112.8

(51) Int. Cl. A61B 5/00; G06F 19/00

(52) U.S. Cl. CPC A61B 5/411; G06F 19/3418; G06F 19/3431; G06F 19/363; G06F 19/3406; G06F 19/325; A61B 5/6801; A61B 5/746; A61B 5/0022; A61B 5/0077; A61B 5/742; A61B 5/749; A61B 5/7275

(57) ABSTRACT

In one embodiment, an apparatus (204) comprising a processor (408) that receives indicators of an allergic reaction, presents an interactive question and answer session (1600) with the user based on the indicators, and provides health information (1620) relating to an allergic reaction.

### CROSS REFERENCE TO RELATED APPLICATION

This application claims the benefit of priority of U.S. Provisional Patent Application Ser. No. 62/130,218, filed on Mar. 9, 2015, and titled “METHODS, SYSTEMS, AND SOFTWARE FOR PROVIDING ALLERGY HEALTH DATA TO A USER,” which is incorporated by reference herein in its entirety.

### FIELD OF THE INVENTION

The present invention generally relates to the field of wearable technology devices.

### BACKGROUND OF THE INVENTION

Many people suffer from various types of allergies, and allergic reactions range from mild, such as itchy eyes and sneezing, to severe, which may include anaphylactic shock and death. Current generation wearable technology devices, such as smartwatches and health-bands, record various physiological data, including data pertaining to physiological parameters corresponding to allergy-related symptoms. For instance, U.S. Patent Application Number 20100087744 describes a wearable pulse rate and rhythm monitoring device that monitors a person's pulse for variables such as pulse rate, pulse rhythm, pulse amplitude and blood pressure versus time when a person is exposed to one or more potential allergens. The person will select a potential allergen, such as corn, from a list stored in the device when the person will be exposed to that particular potential allergen. Selecting a potential allergen will start the device to monitor the pulse for gathering the data and storing the data in the memory for a pre-selected period of time. The stored data is then downloaded into a program that will plot the pulse rate versus real time so that an allergen can be identified. By monitoring the various changes to a person's pulse versus real time instead of taking an average, even slight allergens can be identified.

### SUMMARY OF THE INVENTION

One object of the present invention is that a wearable device can detect indicators of an allergic reaction.

Another object of the present invention is that a wearable device can commence a question and answer session with a user based on the indicators to determine health information related to the allergic reaction.

Another object of the present invention is that a wearable device can provide health information relating to the allergic reaction to a user.

To better address such concerns, in a first aspect of the invention, an apparatus comprising a processor that receives indicators of an allergic reaction, presents an interactive question and answer session with the user based on the indicators, and provides health information relating to an allergic reaction. The present invention addresses a problem in the art where there is a failure to acquire real-time, substantiating feedback of indicators of allergic reaction conditions and no automatic and immediate remedial measures by commencing an interactive question and answer session with the user at the inception of detection of the condition and providing health information that is helpful to the user in remedying the condition. The question and answer session facilitates the determination of the cause and severity of the allergic reaction

In one embodiment, the processor is further configured to, prior to the presenting of the questions: receive an event time that commences a reaction time window as a function of said received indicators; and determine whether or not the reaction time window has closed, wherein when the determination is that the reaction time window has not closed, initiate the presenting of the questions. The event time may comprise a time when the user stops eating, or when the user is stung by a bee, where the reaction time window may be defined from data received by the processor from third party databases, such as medical or research databases. The databases may also include databases of other users and/or health databases (e.g., pollen status, etc.). The event time may differ for different types of allergies and/or intolerances to allergens, and such differences may be manifested in the different databases. Note that the detection of an allergic reaction condition may involve detection of allergens and/or detection of intolerances to one or more allergens. By delaying the commencement of the interactive session until receiving an event time, resources may be conserved by avoiding unnecessary processing, an important consideration for battery or renewable energy-driven wearable devices.

In one embodiment, the processor is further configured to present the questions only if the indicators indicate that the user is experiencing at least two symptoms corresponding to the allergic reaction. The indicators may include physiological symptoms and non-physiological symptoms. By delaying the interactive question and answer session until the user experiences at least two symptoms, the processor conserves energy and mitigates the risk of false alarms.

In one embodiment, the processor is further configured to assign a value to each of the answers received from the user; and generate a total of the values of the answers, wherein the health information is based on the total, which facilitates the determination of the severity of the allergic reaction.

In one embodiment, the processor is further configured to associate the total of the values of the answer to a health-risk level selected among a plurality of available health risk levels, wherein the processor is further configured to provide an alert corresponding to the health-risk level. The association with defined health risk levels and provision of alerts provide a user-friendly and responsive user interface that informs the user of his or her condition in a readily discernible format.

In one embodiment, the processor is further configured to present a suggested remedy, provide the health information to a third party device, or both present the suggested remedy and provide the health information to the third party. For instance, the third party device may be an electronic device for a clinician or medical provider, which may be of particular importance for conditions deemed most severe. The suggested remedy, in today's world of high cost of health services and long wait times for health services, provides an alternative to addressing many conditions that may be treated at home, saving expense, time, and worry.

These and other aspects of the invention will be apparent from and elucidated with reference to the embodiment(s) described hereinafter.

### BRIEF DESCRIPTION OF THE DRAWINGS

Many aspects of the invention can be better understood with reference to the following drawings, which are diagrammatic. The components in the drawings are not necessarily to scale, emphasis instead being placed upon clearly illustrating the principles of the present invention. In certain instances, details that are not necessary for an understanding of the embodiments or that render other details difficult to perceive may have been omitted. Moreover, in the drawings, like reference numerals designate corresponding parts throughout the several views.

FIG. 1 is a flow diagram that illustrates an example method for providing health information to a user that relates to an allergic reaction the user may be experiencing, in accordance with an embodiment of the invention.

FIG. 2 is a schematic diagram of an example wearable allergen analysis system that can be used in performing the method of FIG. 1, in accordance with an embodiment of the invention.

FIG. 3 is a schematic diagram of a matrix representing example relationships between allergic reaction symptoms and data inputs from sensors and questions, in accordance with an embodiment of the invention.

FIG. 4 is a schematic diagram of an example wearable device that can be used to implement one or more aspects of a method of the present disclosure, including the method of FIG. 1, in accordance with an embodiment of the invention.

FIG. 5A is a schematic diagram of example wearable software that can be implemented on a wearable device of the present disclosure, including the wearable device of FIG. 2, in accordance with an embodiment of the invention.

FIG. 5B is a flow diagram that illustrates an example method that may be performed by wearable software of the present disclosure, including the wearable software of FIG. 5A, in accordance with an embodiment of the invention.

FIG. 6 is a flow diagram that illustrates an example method that may be performed by wearable software of the present disclosure, including the specific allergy database loading software of FIG. 5A, in accordance with an embodiment of the invention.

FIG. 7 is a flow diagram that illustrates an example method that may be performed by initial symptom software of the present disclosure, including the initial symptom software of FIG. 5A, in accordance with an embodiment of the invention.

FIG. 8 is a flow diagram that illustrates an example method that may be performed by questionnaire/action software of the present disclosure, including the questionnaire/action software of FIG. 5A, in accordance with an embodiment of the invention.

FIG. 9 is a flow diagram that illustrates an example method that may be performed by risk calculation software of the present disclosure, including the risk calculation software of FIG. 5A, in accordance with an embodiment of the invention.

FIG. 10 is a schematic diagram that illustrates an example wearable symptoms and comparison database that can be used for the wearable symptoms comparison database of FIG. 2, in accordance with an embodiment of the invention.

FIG. 11 is flow diagram that illustrates an example method that may be performed by wearable software of the present disclosure, including the remedy software of FIG. 5A, in accordance with an embodiment of the invention.

FIG. 12 is a schematic diagram that illustrates an example wearable condition and symptom database that can be used for the wearable condition and symptom comparison database of FIG. 2, in accordance with an embodiment of the invention.

FIG. 13 is a schematic diagram that illustrates an example wearable allergy database that contains data specific to an execution of the software of FIG. 5A, in accordance with an embodiment of the invention.

FIG. 14 is a schematic diagram that illustrates an example wearable questionnaire/action database that can be used for the wearable symptoms comparison database of FIG. 2, in accordance with an embodiment of the invention.

FIG. 15 is a screen diagram of an example wearable base graphical user interface (GUI) that can be used as the wearable base GUI of FIG. 2, in accordance with an embodiment of the invention.

FIG. 16 is a screen diagram of an example questionnaire GUI that can be used as the wearable questionnaire GUI of FIG. 2, in accordance with an embodiment of the invention.

FIG. 17 is a screen diagram of an example remedy GUI that can be used as the wearable remedy GUI of FIG. 2, in accordance with an embodiment of the invention.

FIG. 18 is an example overall method that incorporates use of a wearable allergen analysis system of the present disclosure, such as the wearable allergen analysis system of FIG. 2, in accordance with an embodiment of the invention.

FIG. 19 is a high-level schematic diagram of a computing system that can be used to implement any one or more of the methodologies of the present disclosure, in accordance with an embodiment of the invention.

### DETAILED DESCRIPTION OF EMBODIMENTS

Aspects of the present disclosure are directed to wearable technology devices, for example, smartwatches, health-bands, fitness-bands, and smartphones, among others, and combinations thereof, that are enabled to assist their wearers in diagnosing and/or treating allergic reactions to any one or more of a variety of allergens, such as food-based allergens, airborne allergens, medicinal allergens, animal-based allergens, etc. The present inventors have discovered that there is currently no way for wearable technology to interactively participate in such diagnosis and/or treatment. Because some allergic reactions can be highly debilitating and even life-threatening, enabling wearable devices to provide such functionality can allow users of this technology to not only quickly recognize allergic reactions but also take remedial measures, for example, as directed by the wearable devices. As described below in detail, such aspects can be facilitated by various user interfaces (UIs), including graphical UIs (GUIs), and other software features running on one or more of a variety of devices, including wearable technology devices (or simply “wearable devices”), and web servers, among other devices. These broad aspects of the present invention are described below in connection with a variety of specific examples. That said, those skilled in the art will readily understand that the specific examples described are just that, examples that will inform and instruct those skilled in the art about broad features that they can then implement in a plethora of ways using only routine knowledge and skill in the art.

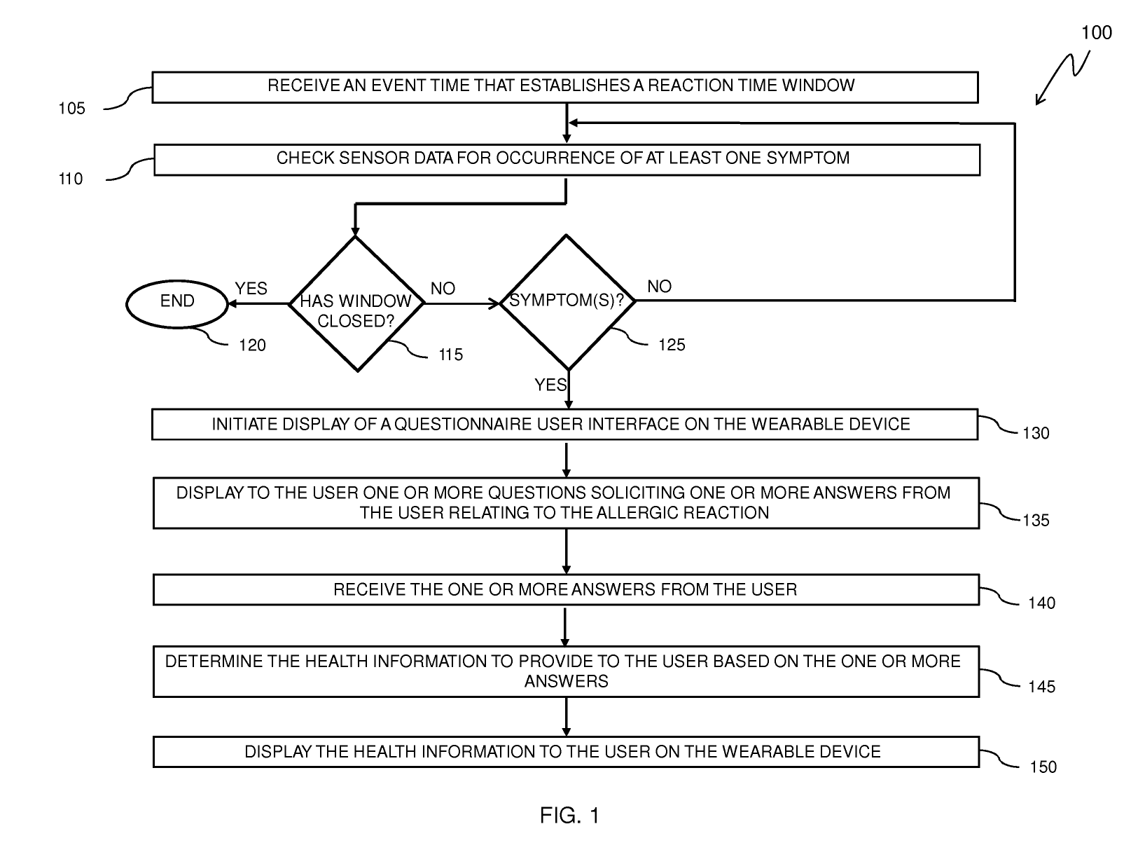

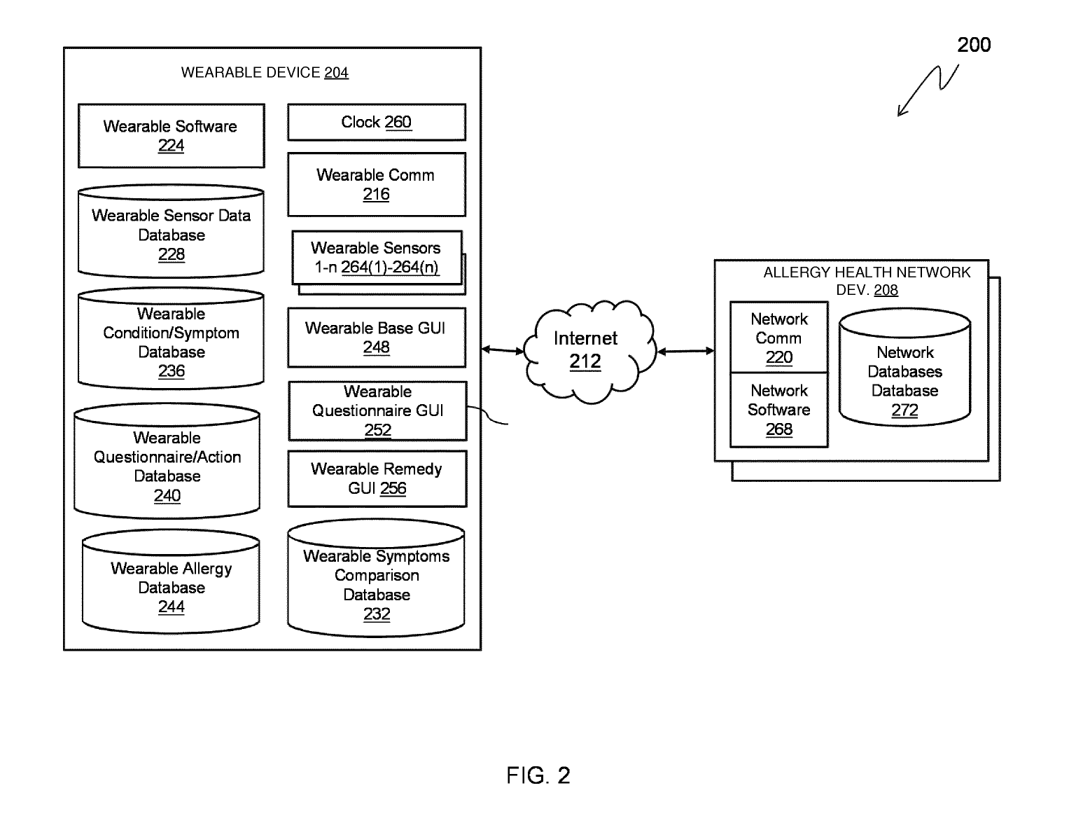

Turning now to the drawings, FIG. 1 illustrates an example high-level method 100 that can be performed by allergen analysis software executed on one or more devices of an allergen analysis system, such as example allergen analysis system 200 of FIG. 2. Before describing method 100 of FIG. 1, allergen analysis system 200 of FIG. 2 is first described to give the reader an example context in which method 100 may be executed. Referring now to FIG. 2, allergen analysis system 200 includes a wearable device 204 and an allergy health network device(s) 208 that communicate via one or more communications networks, here represented by the Internet 212, via corresponding communications systems, here, labeled “Wearable Comm 216” and “Network Comm 220”, respectively. Those skilled in the art will readily understand that each wearable communications system may include, but not be limited to a 3G, 4G, 5G, Wi-Fi, 802.11, visible light, wired, etc., communications system, or any combination thereof. Not shown, but which those skilled in the art will readily understand to be present, are the specific additional communications systems, such as wireless data communications systems (e.g., cellular-based communications systems and satellite-based communications systems, WI-FI™ communications systems, etc.) and wired communications system (e.g., optical fiber based communications systems, copper wire based communications systems, etc.), that work together to provide the point-to-point communications needed between the wearable device 204 and the allergy health network device 208.

In the embodiment shown, the wearable device 204 includes, in addition to Wearable Comm 216, wearable software 224, a wearable sensor database 228, a wearable symptoms comparison database 232, a wearable condition/symptom database 236, a wearable questionnaire/action database 240, a wearable allergy database 244, a wearable base GUI 248, a wearable questionnaire GUI 252, a wearable remedy GUI 256, a clock 260, and one or more sensors 264(1) to 264(N). Note that, although functionality is described herein for a wearable device, including devices worn around the wrist or other body parts or on clothing, one or more of the functionality may be implemented in other devices, including smartphones, personal digital assistants, laptops, among other portable devices in some embodiments. Also in this embodiment, the allergy health network device 208 includes, in addition to the Network Comm 220, network software 268 and a network databases database 272. Each of these components is described and/or referred to in the context of the examples provided below. The allergy health network device 208 may be used and/or associated with any suitable network of allergy experts and/or allergy information providers. As described below in example detail, the allergy health network device 208 may contain information that the wearable device 204 needs to execute allergy-based methodologies of the present disclosure. Such information may include, but not be limited to, symptom data that the wearable device 204 uses to determine whether or not the user is experiencing an allergic reaction, symptom and remedial measures questionnaires, and remedial measures and corresponding data.

Referring again to FIG. 1, the method 100 may begin at step 105 at which the wearable device 204 receives an event time that establishes a reaction time window in which the allergic reaction should occur if it is occurring at all. As an example of such an event time, for an allergic reaction to food, the event time may be selected as the time the user stopped eating. In this example, the wearable device 204 may receive the event time by receiving an input from the user, for example, via the wearable base GUI 248, via sensor input that determines that the user has stopped chewing, or by other means. An another example, if the event is a bee sting, the event time may be determined from a user input to the wearable device 204 of the time, such as via the wearable base GUI 248, or by a microphone sensor detecting the user yelling and/or saying “I've been stung by a bee!,” among other things. The event time may correspond to the detection of an allergen or intolerance to the allergen. At step 110, the wearable device 204 checks sensor data for at least one symptom of a possible allergic reaction being expressed by the user. Note that in some embodiments, the data need not be wearable sensor data, or may include externally-received data in addition to wearable sensor data. For instance, the wearable device 204 may receive (e.g., wirelessly or over a wired connection) data from another device that corresponds to indicators of an allergic reaction, such as imaging data received from an external camera or indirectly via an intermediary device (e.g., revealing physical changes in a user that indicate an allergic reaction), sound data directly or indirectly received from another device (e.g., revealing troubled breathing), among other data. As described in an example below, the symptom(s) the wearable device 204 may check for may be obtained in any suitable manner, such as being downloaded from the allergy health network device 208 of FIG. 2 (and/or in some embodiments, some common symptoms loaded at the time of manufacture of the device 204), for example, in response to the user setting up the wearable device 204 to check for and be responsive to a certain allergic reaction, such as a reaction to a peanut allergy. An example for a peanut allergy is described below. The sensor data that the wearable device 204 checks at step 110 is data obtained from one or more of sensors (FIG. 2) onboard (residing on) the wearable device 204. The sensor data may correspond to one or more sensed physiological parameters, including indicators of a sympathetic reaction (e.g., increased heart rate, increased temperature, breathing frequency changes, blood pressure changes). Note that in some embodiments, data other than physiological data may be received (e.g., as sensed by the wearable sensors and/or communicated by coupled devices) in addition to the physiological data. For instance, the wearable device 204 may receive input corresponding to the environment, such as pollen count or strength, humidity, air quality, mold, etc. As another example, input may be received corresponding to food and/or food composition. That is, sensor devices including smart plates, smart utensils, spectrometers, gluten sensors, among others, may be used to sense the presence of pesticides, gluten, or other allergens. In some embodiments, the sensor devices may include imaging devices and/or code reading technology that read codes (e.g., QR codes, RFID) that may be affixed to food packaging and use accelerometer data (e.g., of arm movement) and/or other sensed data (e.g., sound detected from microphones) to detect the consuming of food. All or a portion of these sensors and sensor data may be utilized by the wearable device 204.

At step 115, the wearable device 204 determines whether or not the reaction time window has closed. If so, then the method 100 may end at 120 or, alternatively, for example, may display a follow-up message to the user indicating the user appears to be safe from an allergic reaction occurring or may continue with monitoring for one or more symptoms for a second allergic reaction. At step 125, the wearable device 204 determines whether or not the user is experiencing the one or more symptoms for the particular allergic reaction that the wearable system is presently programmed to handle. If the wearable device 204 does not determine that the required symptom(s) is/are present at step 125, the method 100 simply loops back to step 110 and keeps checking for the symptom(s) until either the reaction time window has closed or the wearable device 204 determines that the symptom(s) is/are present.

If at step 125 the wearable device 204 determines that the one or more requisite symptoms are present, at step 130 the wearable device 204 displays a questionnaire user interface, such as wearable questionnaire GUI of FIG. 2. In some embodiments, the initiation of the questionnaire (and presentation) is automatic (e.g., without user intervention), based on the sensor data. At step 135, the wearable device 204 displays to the user via the questionnaire user interface one or more questions soliciting one or more answers from the user relating to the allergic reaction under consideration. For example, each such question may be devised to elicit a response from the user that supplements the sensor data that the wearable device 204 checked at step 110 and determined to indicate that the user may be experiencing an allergic reaction of the type that the wearable device 204 is checking for. Having additional data from the answers the user provides to the question(s) asked at step 135 enables the wearable device 204 to make a better informed decision about whether or not the allergic reaction is truly occurring and its severity, since the physiological condition(s) of the sensor data at steps 110 and 125 may be due to something other than the allergic reaction.

At step 140, the wearable device 204 receives the one or more answers the wearable system solicited at step 135, and at step 145 the wearable device 204 determines the health information to provide to the user based on the one or more answers the wearable system receives via the questionnaire user interface. Examples of health information that the wearable device 204 may provide to the user are described below in connection with a detailed example. In one embodiment, the health information may include an identification or confirmation of the occurrence of an allergen reaction (e.g., including an alert), an identity of the cause and/or type of the allergic reaction and/or its severity, and/or suggested mitigating and/or remedial measures or actions plans to take. The remedial/mitigating measures may be based on the severity of the allergic reaction. In some embodiments, the health information may include am elimination diet along with coaching (e.g., textual, graphical, and/or audible) to assist the user in arranging meals in a manner that temporarily or permanently removes different types of foods and/or ingredients from the diet of the user. By doing so, the user may be made aware of which foods and/or their component parts cause an allergic reaction by the user, and take steps to ensure the source of the allergic reaction is avoided. At step 150, the wearable device 204 may display the health information to the user via a suitable remedy UI, such as wearable remedy GUI 256 of FIG. 2. The method 100 of FIG. 1 is exemplified by detailed examples provided below, as are the various features and functionalities of example allergen analysis system 200 of FIG. 2. Note that in some embodiments, all or a portion of the steps above may be implemented on another device or devices, such as a user device (e.g., smartphone, personal digital assistant, etc.) and/or a network device (e.g., remote server).

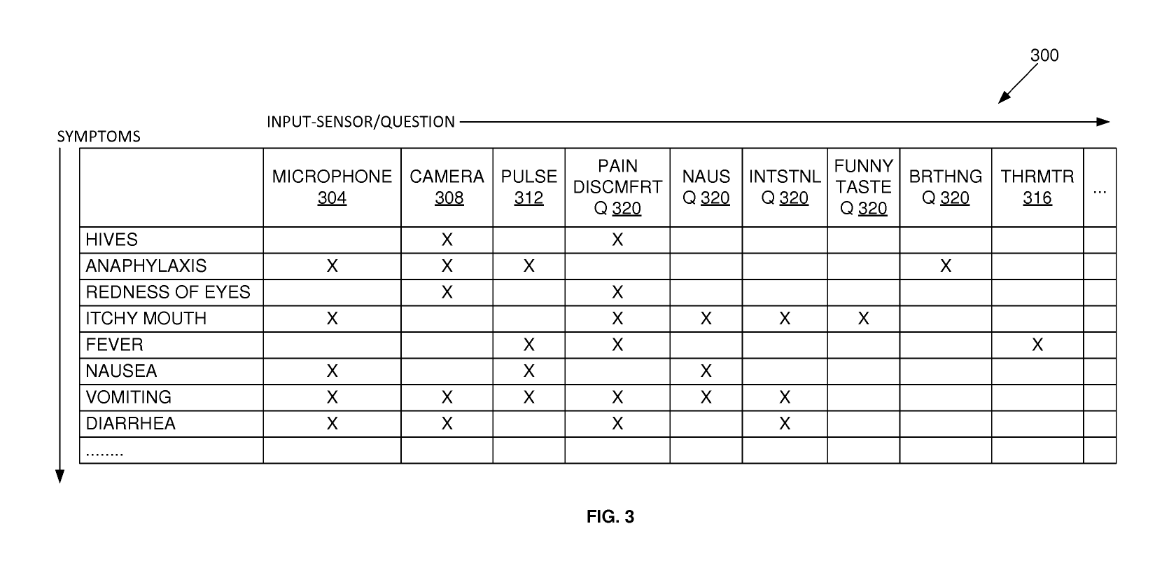

Before describing an example that illustrates the method 100 of FIG. 1 and the example allergen analysis system 200 of FIG. 2 in more detail, attention is directed to FIG. 3, which illustrates an example symptoms-versus-data-input matrix 300 that shows the types of data inputs that the wearable device 204 can use to determine whether or not a particular symptom of an allergic reaction is present. In this example, example sensors 264(1)-264(n) that may be located onboard (e.g., wearable sensors) the wearable device 204 and/or located externally yet in communication with the wearable device 204 are a microphone 304, a camera 308, a pulse sensor 312, and a thermometer 316. Note that fewer, additional, and/or different sensors may be used in some embodiments. For instance, in some embodiments, the camera 308 and/or the microphone 304 may be omitted. In some embodiments, the sensors 264 may include biomolecular detection devices that sense target markers present in bodily fluid, including blood, serum, plasma, lymph, perspiration, saliva, tears, and/or urine. The sensors 264 may include sensors that utilize infrared imaging of the blood (e.g., to detect the manifestation of histamines), skin conductivity or impedance, blood pressure sensors. For instance, imaging data may be used in combination with image recognition software residing in the wearable device 204 (or the results of image capture and recognition residing elsewhere and communicated to the wearable device 204) to detect certain foods that are known allergens and/or detect physiological conditions of the user known to be suspected manifestations of an allergic reaction, such as detection of rashes or eczema, welling of the lips, face, or other parts of the body. In some embodiments, microphones on the wearable device 204 (or residing elsewhere with corresponding data communicated to the wearable device 204) may be used alone or in conjunction with the other sensors to detect any manifestation of an allergic reaction, including wheezing, nasal congestion, breathing irregularities, diarrhea, nausea, vomiting. The questions may be presented to solicit answers to substantiate the sensor data and/or to help reveal whether or not an allergic reaction is occurring where the sensor data is unavailable or insufficient to detect some allergens and/or allergic reactions. In other words, other data input (beyond the sensor data) may be obtained via a suitable questionnaire that is presented to the user via a questionnaire UI, such as the wearable questionnaire GUI 252 of FIG. 2. In this example, the questions presented by wearable questionnaire GUI 252 include a pain/discomfort question 320, a nausea question 324, an intestinal question 328, a funny-taste question 332, and a breathing question 336, each designed to elicit a response from the user that provides more data to assist wearable device 204 in determining whether or not one or more of the relevant symptoms are present. Those skilled in the art will readily understand that the contents of symptoms-versus-data-input matrix 300 is merely one example and non-limiting, since other symptoms, sensors, and questions may be used.

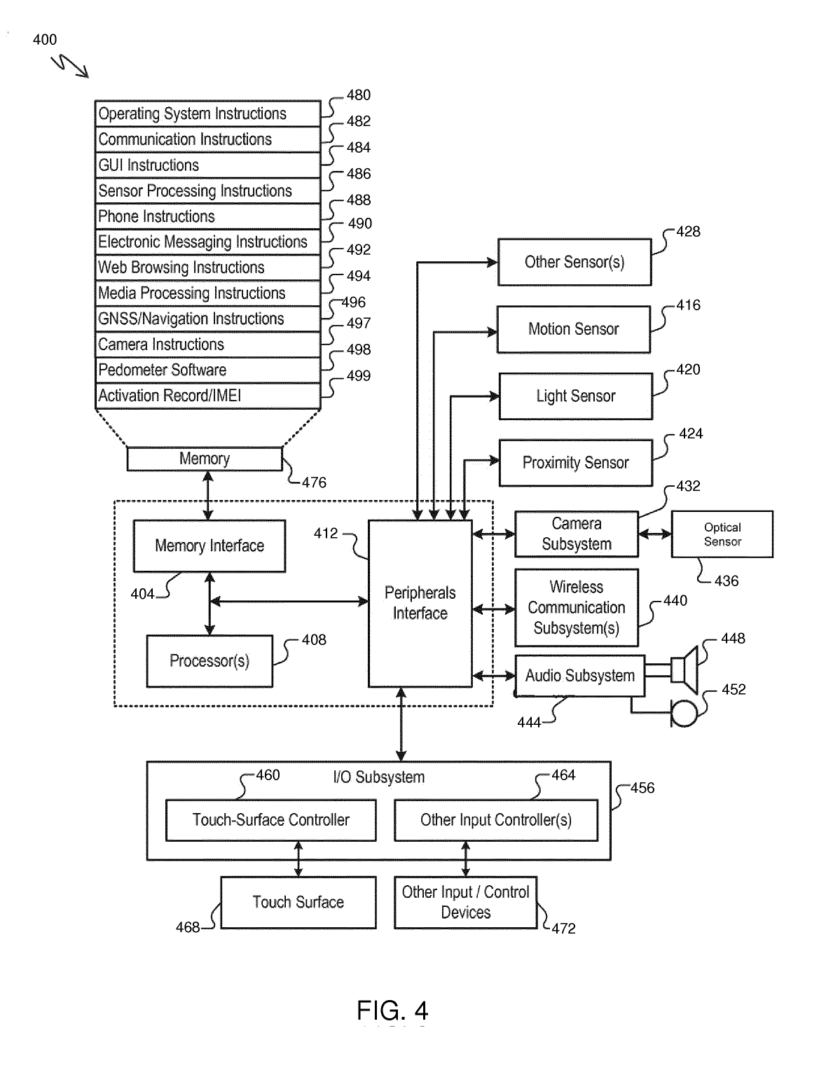

FIG. 4 is a block diagram of an example wearable computing device 400 that may be configured to implement any one or more of various features and/or processes of the present disclosure, such as the features and processes illustrated in other figures of this disclosure, as well as features and processes that would be apparent to those of ordinary skill in the art after reading this entire disclosure. As shown, the computing device 400 may include a memory interface 404, one or more data processors, image processors and/or central processing units 408, and a peripherals interface 412. Memory interface 404, one or more processors 408, and/or peripherals interface 412 may be separate components or may be integrated in one or more integrated circuits. The various components in computing device 400 may be coupled by one or more communication buses or signal lines.

Sensors, devices, and subsystems may be coupled to peripherals interface 412 to facilitate one or more functionalities. For example, a motion sensor 416, a light sensor 420, and a proximity sensor 424 may be coupled to peripherals interface 412 to facilitate orientation, lighting, and/or proximity functions. Other sensors 428 may also be connected to peripherals interface 412, such as a global navigation satellite system (GNSS) (e.g., GPS receiver), a temperature sensor, a biometric sensor (e.g., sensing physiological or behavioral parameters), and/or one or more other sensing devices, to facilitate related functionalities, including environmental and/or food detection.

A camera subsystem 432 and an optical sensor 436, e.g., a charged coupled device (CCD) or a complementary metal-oxide semiconductor (CMOS) optical sensor, may be utilized to facilitate camera functions, such as recording images and/or video. Camera subsystem 432 and optical sensor 436 may be used to collect images of a user to be used during authentication of a user, e.g., by performing facial recognition analysis. For instance, as set forth above, the camera subsystem 432 may be used to detect physical changes in the skin and/or body that evidences an allergic reaction. In some embodiments, the camera subsystem 432 may be used to detect food and/or its components (e.g., including types of food that are suspected allergens, pesticides, etc.).

Communication functions may be facilitated through one or more wireless communication subsystems 440, which may include radio frequency receivers and transmitters and/or optical (e.g., infrared) receivers and transmitters. The specific design and implementation of communication subsystem 440 may depend on the communication network(s) over which computing device 400 is intended to operate. For example, computing device 400 may include communication subsystems 440 designed to operate over a GSM network, a GPRS network, an EDGE network, a Wi-Fi™ or WiMax™ network, and/or a Bluetooth™ network. The communication subsystem 440 may use near field communication technology and/or RFID or other readable code technology to identify types of food that are to be consumed. In particular, wireless communication subsystems 440 may include hosting protocols such that one or more devices 400 may be configured as a base station for other wireless devices.

An audio subsystem 444 may be coupled to a speaker 448 and a microphone 452 to facilitate voice-enabled functions, such as speaker recognition, voice replication, digital recording, and/or telephony functions. Audio subsystem 444 may be configured to facilitate processing voice commands, voice-printing, and voice authentication. The audio subsystem 444 may also be used to detect audible manifestations of an allergic reaction, including wheezing, coughing, diarrhea, troubled and/or irregular breathing, falls to the ground, bee stings, etc.

I/O subsystem 456 may include a touch-surface controller 460 and/or other input controller(s) 464. Touch-surface controller 460 may be coupled to a touch surface 468. Touch surface 468 and touch-surface controller 460 may, for example, detect contact and movement or a lack thereof using one or more of any of a plurality of touch sensitivity technologies, including but not limited to capacitive, resistive, infrared, and/or surface acoustic wave technologies, optionally as well as other proximity sensor arrays and/or other elements for determining one or more points of contact with touch surface 468.

Other input controller(s) 464 may be coupled to other input/control devices 472, such as one or more buttons, rocker switches, thumb-wheel, infrared port, USB port, and/or a pointer device such as a stylus. One or more related buttons or other controls (not shown) may include one or more sets of up/down buttons for volume and/or amplitude control of speaker 448 and/or microphone 452. Using the same or similar buttons or other controls, a user may activate a voice control, or voice command, module that enables the user to speak commands into microphone to cause device 400 to execute the spoken command. The user may customize functionality of one or more buttons or other controls. Touch surface 468 may, for example, also be used to implement virtual or soft buttons and/or a keyboard.

In some implementations, computing device 400 may present recorded audio and/or video files, such as MP3, AAC, and/or MPEG files. In some implementations, computing device 400 may include the functionality of an MP3 player, such as an iPod™. Computing device 400 may, therefore, include a 36-pin connector that is compatible with related iPod™ hardware. Other input/output and control devices may also be used.

As shown, memory interface 404 may be coupled to one or more types of memory 476. Memory 476 may include high-speed random access memory and/or non-volatile memory, such as one or more magnetic disk storage devices, one or more optical storage devices, and/or flash memory (e.g., NAND, NOR). Memory 476 may store an operating system 480, such as Darwin™ RTXC, LINUX, UNIX, OS X™, WINDOWS™, and/or an embedded operating system such as VxWorks. Operating system 480 may include instructions for handling basic system services and/or for performing hardware dependent tasks. In some implementations, operating system 480 may comprise a kernel (e.g., UNIX kernel). Further, in some implementations, operating system 480 may include instructions for performing voice authentication.

Memory 476 may also store communication instructions 482 to facilitate communicating with one or more additional devices, one or more computers, and/or one or more servers. Additionally or alternatively, memory 476 may include: graphical user interface instructions 484 to facilitate graphic user interface processing; sensor processing instructions 486 to facilitate sensor-related processing and functions; phone instructions 488 to facilitate phone-related processes and functions; electronic messaging instructions 490 to facilitate electronic-messaging related processes and functions; web browsing instructions 492 to facilitate web browsing-related processes and functions; media processing instructions 494 to facilitate media processing-related processes and functions; GNSS/Navigation instructions 496 to facilitate GNSS and navigation-related processes and instructions; and/or camera instructions 497 to facilitate camera-related processes and functions. Memory 476 may store other software instructions 498 to facilitate other processes and functions. For example, other software instructions 498 may include instructions for counting steps the user takes when device 400 is worn.

Memory 476 may also store other software instructions (not shown), such as web video instructions to facilitate web video-related processes and functions and/or web shopping instructions to facilitate web shopping-related processes and functions. In some implementations, media processing instructions 494 may be divided into audio processing instructions and video processing instructions to facilitate audio processing-related processes and functions and video processing-related processes and functions, respectively. An activation record and International Mobile Equipment Identity (IMEI) 499 or similar hardware identifier may also be stored in memory 476.

Each of the above identified instructions and applications may correspond to a set of instructions for performing one or more functions described herein. These instructions need not necessarily be implemented as separate software programs, procedures, or modules. Memory 476 may include additional instructions or fewer instructions. Further, various functions of computing device 400 may be implemented in hardware and/or in software, including in one or more signal processing and/or application specific integrated circuits.

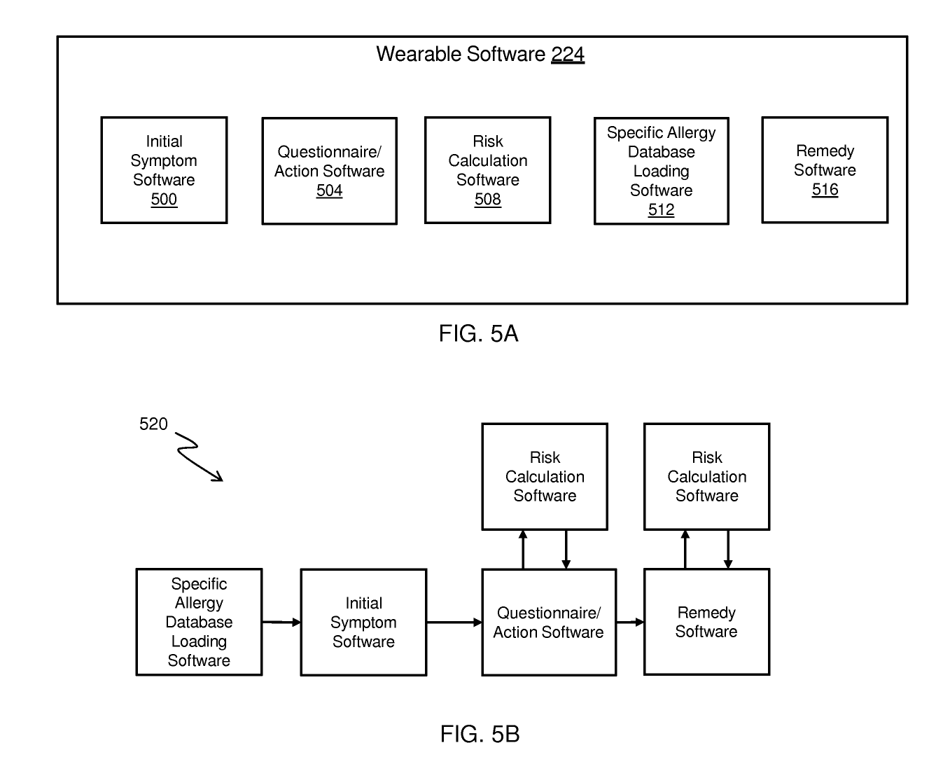

Referring now to FIGS. 5A and 5B, FIG. 5A illustrates an example of the wearable software 224 of the wearable device 204 of FIG. 2, including a number of software components, namely, initial symptom software 500, questionnaire/action software 504, risk calculation software 508, specific allergy database loading software 512, and remedy software 516, each of which is described in more detail below. It is noted that the term “software” as used to describe any of the software herein does not necessarily connote any particular arrangement or compartmentalization or segmentation of software code. Rather, the term is simply used in conjunction with a descriptor to indicate the functionality the software provides. FIG. 5B is a flow diagram 520 illustrating the general flow of execution of component software 500-516 of wearable software 204, including a specific allergy database loading software, initial symptom software, questionnaire/action software in cooperation with risk calculation software, and remedy software in cooperation with risk calculation software.

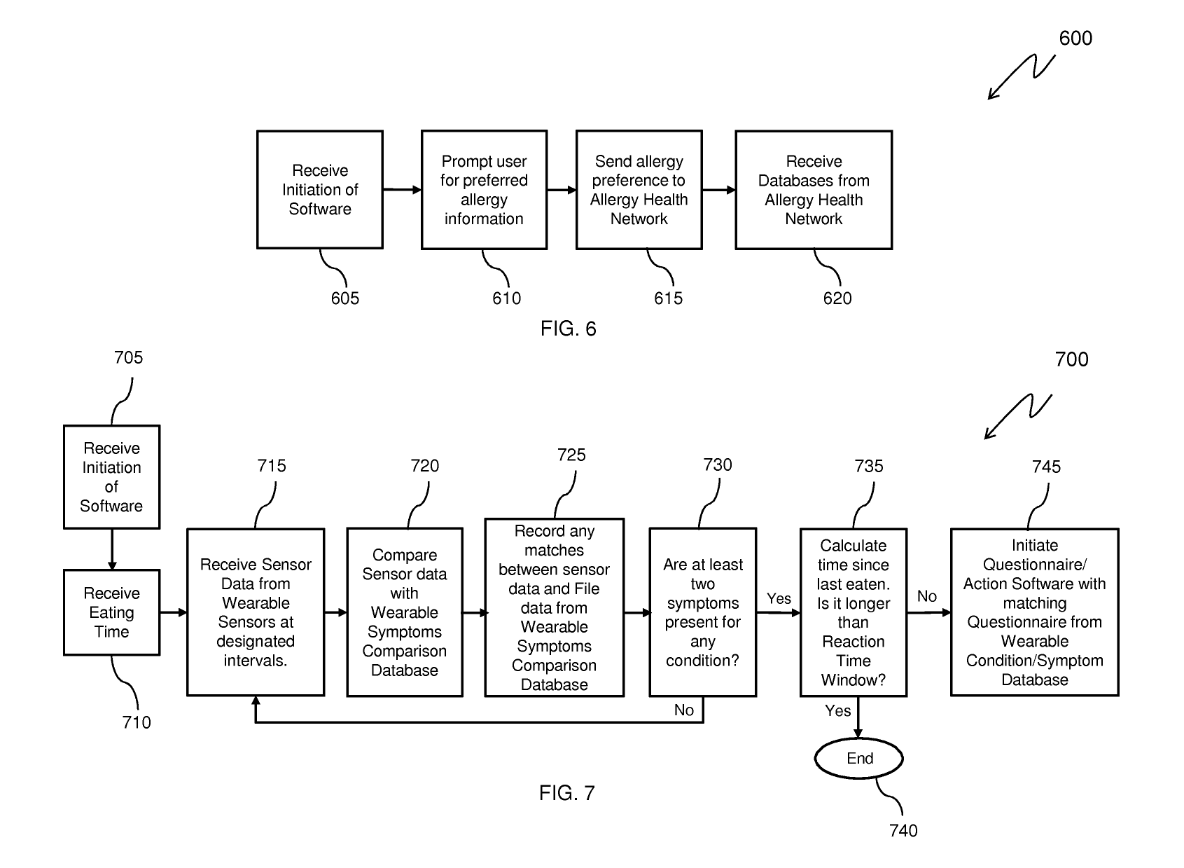

FIG. 6 illustrates an example method 600 that specific allergy database loading software 512 of FIGS. 5A-5B may perform. As seen in FIG. 6, the method 600 receives an initiation of specific allergy database loading software 512 (step 605), prompts for and receives from a user preferred allergy information (step 610), sends the allergy preference to allergy health network device 208 of FIG. 2 (step 615), and receives the corresponding databases, such as wearable condition database 236, wearable questionnaire/action database 240, wearable symptoms comparison database 232, and wearable allergy database 244, from the allergy health network device (step 620). This allows the user to have the wearable device 204 monitor the user for the occurrence of an allergic reaction to a particular allergen.

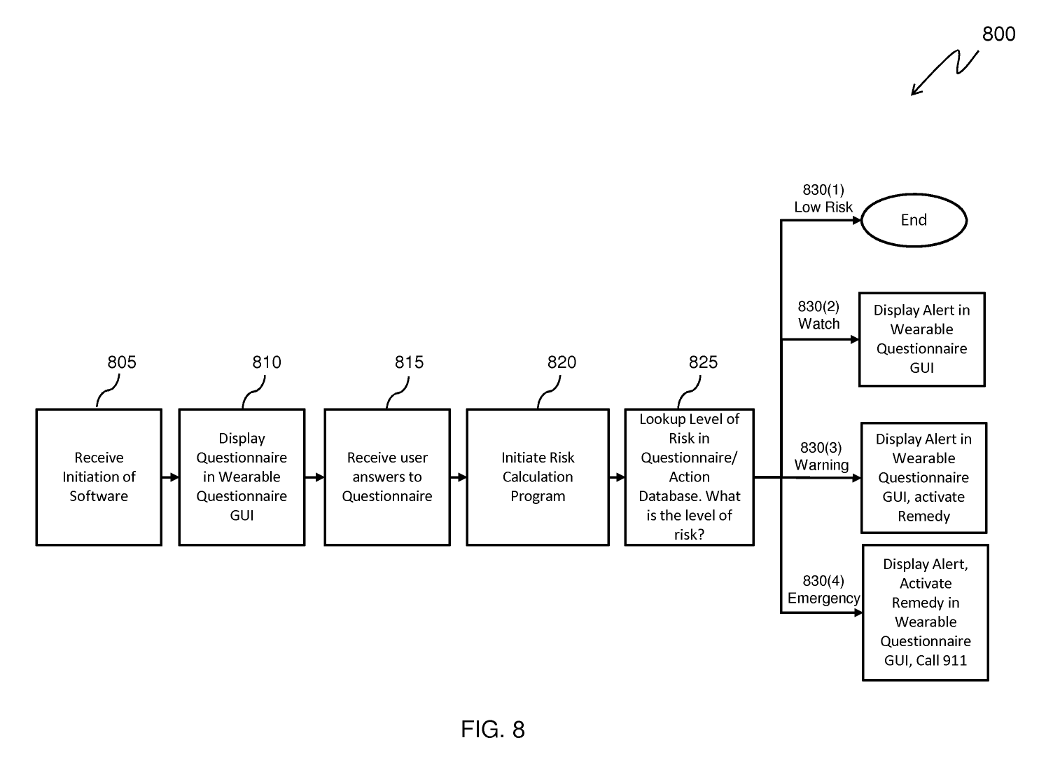

FIG. 7 illustrates an example method 700 that the initial symptom software 500 of FIGS. 5A-5B may perform. As seen in FIG. 7, the method 700 receives an initiation of initial symptom software 500 (step 705), receives an eating time, i.e., an event time, here directed to a food allergy (step 710), receives sensor data, at designated intervals, from one or more of the sensors 264(1)-264(n) of FIG. 2 relevant to the allergy at issue from the method 600 (step 715), compares the sensor data with wearable symptoms comparison database 232 (step 720), and records matches between the sensor data and file data from wearable symptoms comparison database 232 (step 725). At step 730, the method 700 determines whether or not the sensor readings indicate that at least two symptoms of the allergy at issue are present in the user. If not, the method 700 loops back to step 715. If so, method 700 proceeds to step 735 at which initial symptom software 500 calculates the time that has elapsed since the eating time received at step 710 to determine whether or not it is longer than a reaction time window that is received from allergy health network 208 as part of the method 600 of FIG. 6. If the time since eating exceeds the reaction time window, then the method 700 may end at step 740. However, if the time since eating is less than the reaction time, then the method 700 may proceed to step 745 at which the initial symptom software 500 initiates questionnaire/action software 504 (FIGS. 5A and 5B) with a questionnaire from wearable condition/symptom database 236 that matches the allergy at issue.

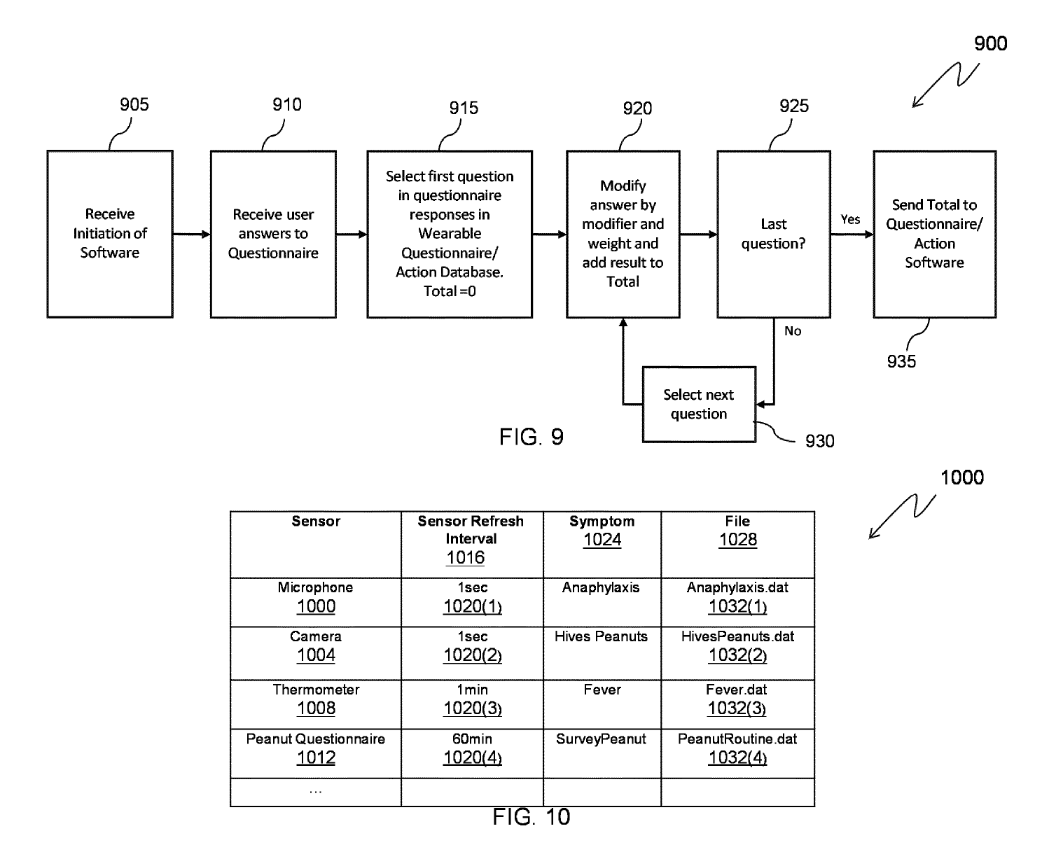

FIG. 8 illustrates an example method 800 that questionnaire/action software 504 of FIGS. 5A-5B may perform. As seen in FIG. 8, the method 800 receives an initiation of questionnaire/action software 504 (step 805), displays the questionnaire noted in step 745 of the method 700 of FIG. 7 in wearable questionnaire GUI 252 (step 810), receives from the user answers to the questionnaire via the wearable questionnaire GUI 252 (step 815), initiates risk calculation software 508 (FIGS. 5A, 5B, and 9) (step 820), looks up a level of risk based on the answers to the questionnaire in questionnaire/action database 240 (step 825), and, depending on the level of risk, takes a corresponding action, here, actions 830(1), 830(2), 830(3), and 830(4) corresponding respectively to risk levels “Low Risk”, “Watch”, “Warning”, and “Emergency”.

FIG. 9 illustrates an example method 900 that risk calculation software 508 of FIGS. 5A-5B may perform. As seen in FIG. 9, the method 900 receives an initiation of risk calculation software 508 (step 905), receives the user's answers to the questionnaire (step 910), selects the first question in the questionnaire responses in wearable questionnaire/action database 240 and sets an accumulator (“Total”) to zero (step 915), modifies the answer by a modifier and a weight and adds the result to the Total (step 920), and determines whether or not the question just modified was the last question (step 925). If the question just modified at step 920 was not the last question at step 925, then method 900 selects the next questions at step 930 and loops back to step 920. If, however, the question modified at step 920 is determined to be the last question at step 925, then method 900 advances to step 935 at which the risk calculation software 508 returns to step 825 of questionnaire/action software 504 (FIG. 8) to determine the level of risk based on the overall total just calculated at step 920 of the method 900 of FIG. 9.

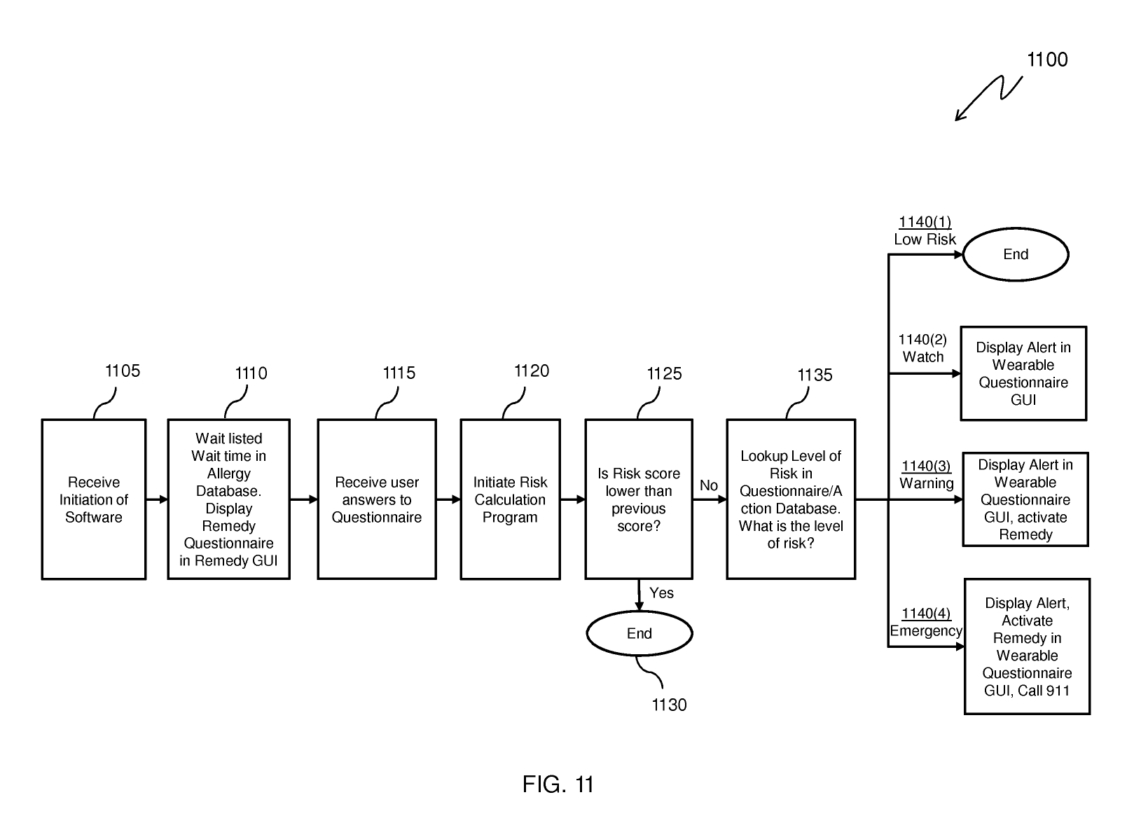

FIG. 10 illustrates example contents for wearable symptoms comparison database 232 of FIG. 2. In this example and as seen in FIG. 10, the subject allergy is a peanut allergy that utilizes three sensors, here a microphone 1000, a camera 1004, and a thermometer 1008 (which can be some or all of sensors 264(1)-264(n) of wearable device 204 of FIG. 2) and a peanut-allergy-specific questionnaire 1012. As seen in FIG. 10, for each of these data inputs, wearable symptoms comparison database 232 contains a refresh interval column 1016 containing values, here values 1020(1) and 1020(2) of 1 second each for microphone 1000 and camera 1004, a value 1020(3) of 1 minute for thermometer 1008, and a value 1020(4) of 60 minutes for peanut questionnaire 1012. In the detailed example, the wearable software 224 (FIGS. 2, 5A, and 5B) uses values 1020(1) to 1020(3) at step 715 of the method 700 of FIG. 7 and value 1020(4) at step 1110 of method 1100 of FIG. 11. The wearable symptoms comparison database 232 also includes a column 1024 designating the symptom relevant to the corresponding sensor, as well as column 1028 containing file identifiers, here file identifiers 1032(1) to 1032(4), for corresponding files containing data to which wearable device 204 compares actual measurements, readings, photographs, answers etc., the wearable system acquires in order to determine whether or not the user is likely to be experiencing an allergic reaction. In the detailed example, wearable software 224 (FIGS. 2, 5A, and 5B) uses data in the files identified by file identifiers 1032(1) to 1032(3) at steps 720 to 730 of method 700 of FIG. 7 and data in the file identified by file identifier 1032(4) at step 810 of the method 800 of FIG. 8.

FIG. 11 illustrates an example method 1100 that the remedy software 516 of FIGS. 5A-5B may perform. As seen in FIG. 11, the method 1100 receives an initiation of remedy software 516 (step 1105), waits for a wait time listed in allergy database 244 and then displays a corresponding remedy questionnaire in remedy GUI 256 of FIG. 2 (step 1110), receives from the user answers to the questionnaire via the remedy GUI 256 (step 1115), and initiates risk calculation software 508 (FIGS. 5A, 5B, and 9) to determine a new risk score (step 1120). At step 1125, the method 1100 determines whether the new risk score is lower than the previous risk score from method 800. If so, the method 1100 proceeds to step 1130 at which the method ends. However, if the new risk score is not lower than the previous risk score, the method 1100 proceeds to step 1135 to look up a level of risk, based on the new risk score, in the questionnaire/action database 240. Depending on the level of risk, the method 1100 takes a corresponding action, here, actions 1140(1), 1140(2), 1140(3), and 1140(4) corresponding respectively to risk levels “Low Risk”, “Watch”, “Warning”, and “Emergency”.

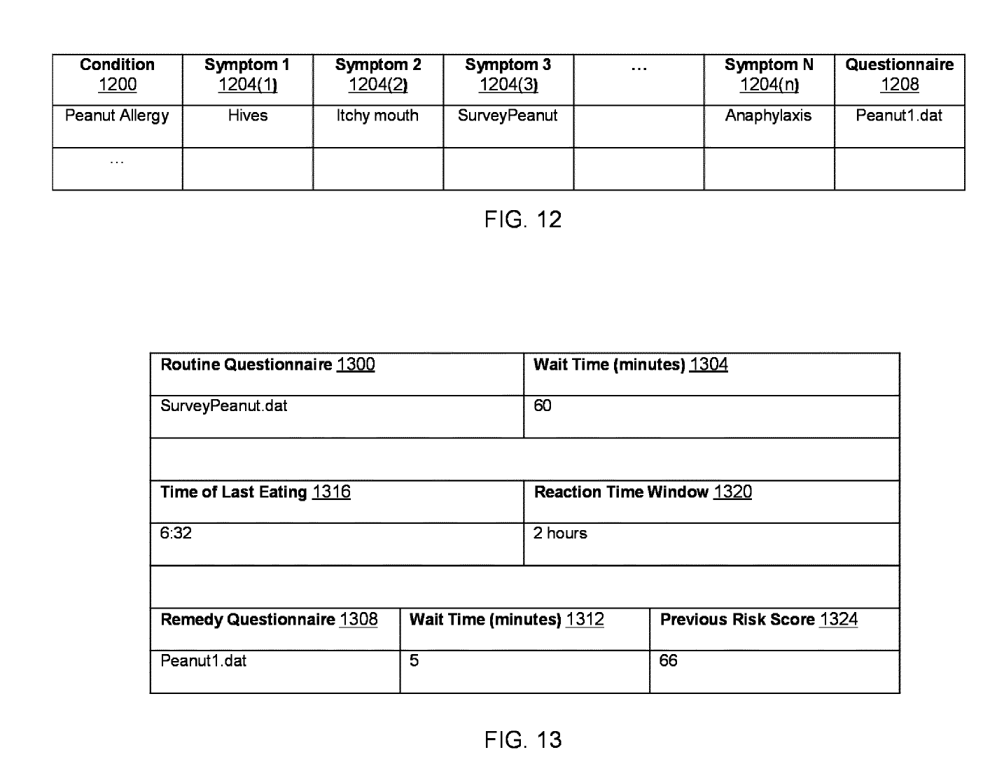

FIG. 12 illustrates example contents of the wearable condition/symptom database 236 of the wearable device 204 of FIG. 2. In this example and as seen in FIG. 12, the wearable condition/symptom database 236 contains the condition (allergy) 1200 along with its corresponding symptoms, here symptoms 1204(1) to 1204(N), and a file identifier 1208 for the pertinent remedy questionnaire.

FIG. 13 illustrates example contents of the wearable allergy database 244 of the wearable device 204 of FIG. 2. In this example, the wearable allergy database 244 contains a file identifier 1300 for the file containing the “routine” or symptom questionnaire used at step 810 of the method 800 of FIG. 8, along with a corresponding wait time 1304. The wearable allergy database 244 also contains a file identifier 1308 for the file containing the remedy questionnaire used at step 1110 of the method 1100 of FIG. 11, along with a corresponding wait time 1312. In addition, the wearable allergy database 244 contains a time 1316 of last eating from step 710 of the method 700 of FIG. 7, a reaction time window value 1320, which wearable device 204 uses at step 735 of the method 700 of FIG. 7, and a previous risk score 1324 calculated in the method 900 of FIG. 9. Those skilled in the art will readily appreciate that the contents of the wearable allergy database 244 are merely one example and are not limiting in any way.

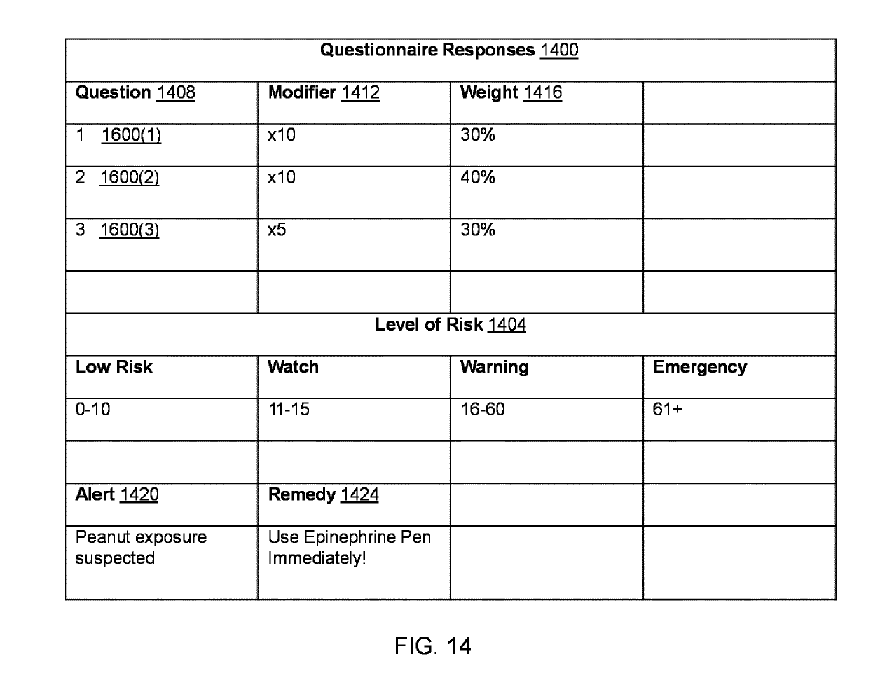

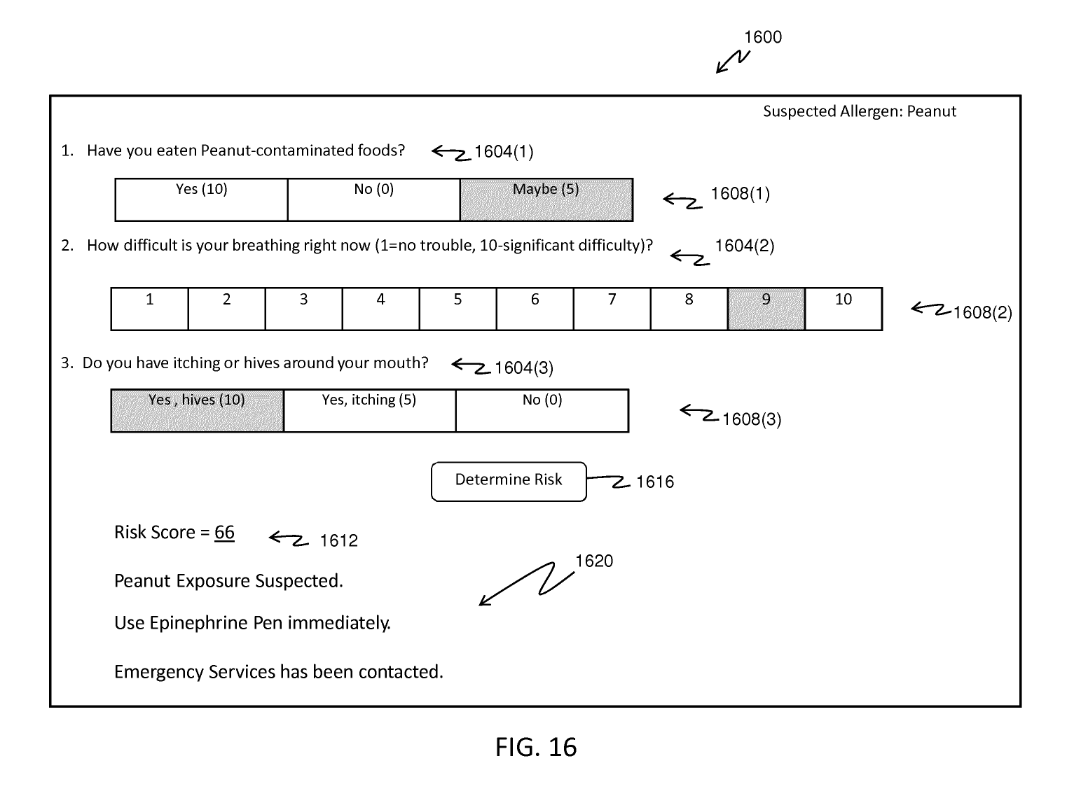

FIG. 14 illustrates example contents of the wearable questionnaire/action database 240 of FIG. 2. The wearable questionnaire/action database 240 may include questionnaire responses data 1400 and level of risk data 1404. The questionnaire responses data 1400 may include the question numbers 1408, corresponding respective modifiers 1412, and corresponding respective weights 1416. As seen in the example questionnaire GUI 252 of FIG. 2 illustrated in FIG. 16, answers to each question 1600(1) to 1600(3) have assigned values, and risk calculation software 508 (FIGS. 5A and 5B) uses the corresponding respective modifiers 1412 and weights 1416 at step 920 of the method 900 of FIG. 9 to calculate a risk score 1612. For example, a user may be asked if they have eaten peanut-contaminated foods. The answer may be worth five points and that may be multiplied by 10 to be 50, which then may be weighted by 30 percent for a resulting total of 15. This may be stored in the questionnaire/action database 240. The weighted and modified scores are totaled up and that total is associated with the level of risk. Level of risk data 1404 in this example contains four levels of risk determined by the total risk calculated at by method 900 of FIG. 9. For example, for a peanut allergy, a total calculated risk of 0-10 is considered low risk, a total calculated risk of 11-15 is in the watch category, a total calculated risk of 16-60 is in the warning category, and a total calculated risk of 61 or higher is in the emergency category. Also shown contained in the wearable questionnaire/action database 240 of FIG. 14 are an alert 1420 and remedy 1424 that wearable device 204 (FIG. 2) may display to the user via remedy GUI 256 as appropriate for the calculated risk. Here the alert 1420 is “Peanut exposure suspected” and remedy 1424 is “Use Epinephrine Pen Immediately!”.

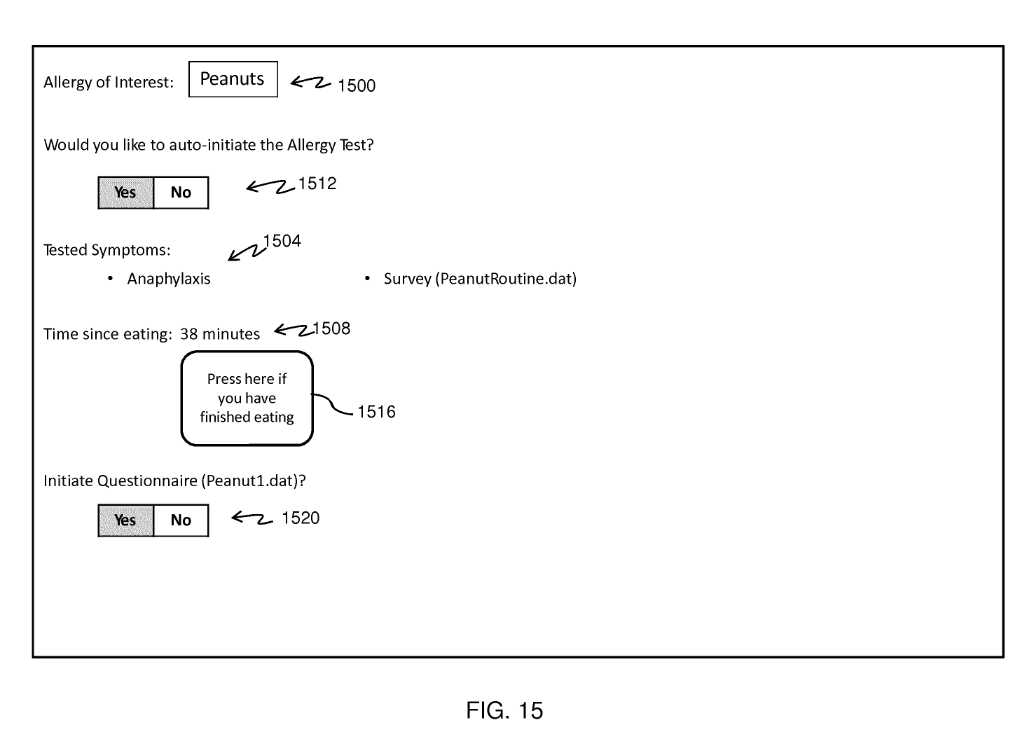

FIG. 15 illustrates an example instantiation of the wearable base GUI 248 of the wearable device 204 of FIG. 2 that is directed to a peanut allergy and corresponding allergic reaction. In this example, the wearable base GUI 248 includes an allergy selector 1500, which here shows that a user has selected “Peanuts”. Once the user has selected the desired allergy, the wearable device 204 causes the wearable base GUI 248 to display specific information pertinent to peanut allergy, here in “Tested Symptoms” region 1504 and in “Time since eating” region 1508. The wearable base GUI 248 also includes a yes/no selector 1512 that allows the user to control whether or not the wearable device 204 auto-initiates allergy testing. If the user selects “Yes” on selector 1512, then the wearable software 224 of FIGS. 2, 5A, and 5B executes automatically (e.g., automatically runs). If the user selects “No” on selector 1512, then the wearable software 224 can only be initiated manually. The wearable base GUI 248 includes an event time input means, here a soft button 1516, which a user selects to indicate when they have stopped eating. The event time is based on clock 260 of the wearable device 204 of FIG. 2, and the wearable device 204 uses this time in various methods, such as the method 700 of FIG. 7 as described above. In this example, the wearable base GUI 248 allows a user to initiate the remedy questionnaire manually, here using a yes/no selector 1520.

As mentioned above, FIG. 16 illustrates an example instantiation of questionnaire GUI 252 of wearable device 204 that the wearable system displays to a user, for example, at step 810 of the method 800 of FIG. 8 to display a questionnaire 1600. As seen in FIG. 16, the questionnaire 1600 includes three questions 1604(1), 1604(2), and 1604(3) and corresponding answer soft controls 1608(1), 1608(2), and 1608(3). As readily seen, each of the answers in answer soft controls 1608(1), 1608(2), and 1608(3) is assigned a numerical value, which the wearable software 224 utilizes in calculating an overall risk score 1612 as described above. In the embodiment shown, the questionnaire GUI 252 includes a “Determine Risk” soft button 1616 that allows the user to initiate the risk score calculation when done answering questions 1604(1), 1604(2), and 1604(3). The questionnaire GUI 252 further includes a message region 1620 in which wearable software 224 displays various messages (e.g., health information) based on the overall risk score 1612.

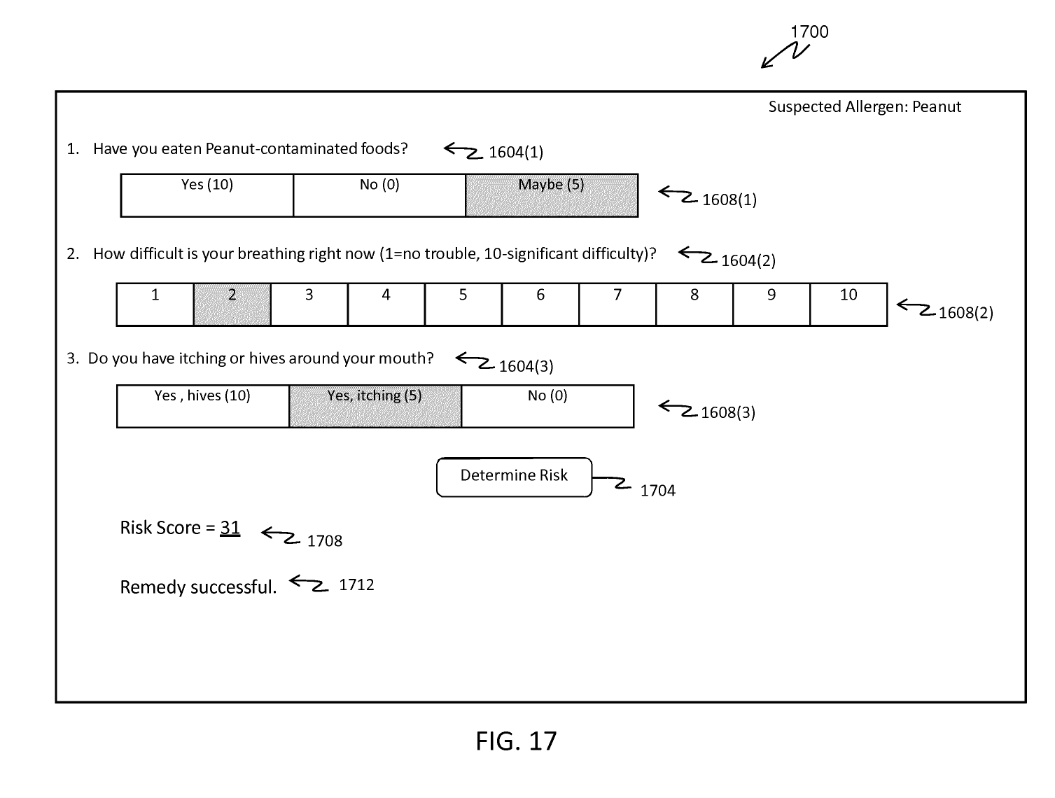

FIG. 17 illustrates an example instantiation of a remedy GUI 256 of the wearable device 204 that the wearable device 204 displays to a user, for example, at step 1110 of the method 1100 of FIG. 11 to display a questionnaire 1700. As seen in FIG. 17, the questionnaire 1700 repeats the three questions 1604(1), 1604(2), and 1604(3) of questionnaire 1600 of FIG. 16 and corresponding answer soft controls 1608(1), 1608(2), and 1608(3). In the embodiment shown, the remedy GUI 256 includes a “Determine Risk” soft button 1704 that allows the user to initiate the risk score calculation when done answering questions 1604(1), 1604(2), and 1604(3) to calculate a post-remedy overall risk score 1708. Remedy GUI further includes a message region 1712 in which the wearable software 224 displays various messages based on post-remedy overall risk score 1708.

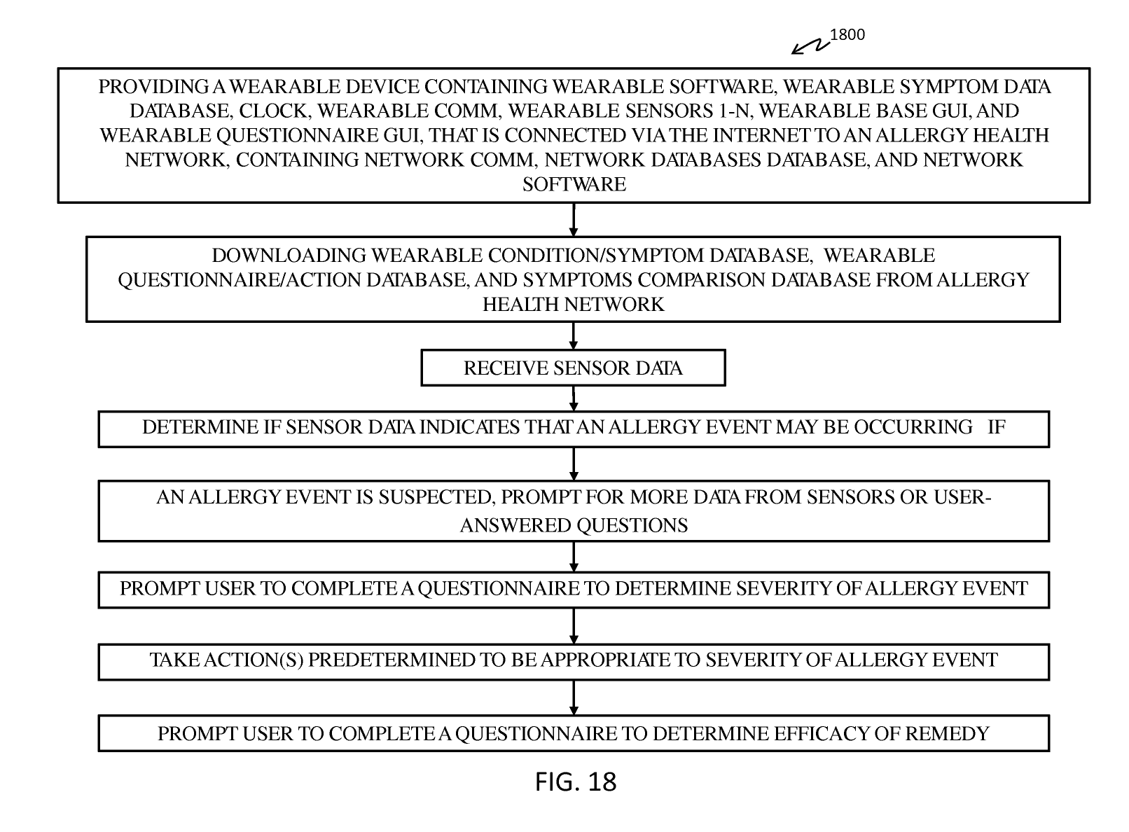

FIG. 18 illustrates an overall method 1800 of utilizing the allergen analysis system 200 of FIG. 2. As seen, the steps of method the 1800 of FIG. 18 are quite self-explanatory when read in the context of the allergen analysis system 200 of FIG. 2 and the detailed examples described above. Those skilled in the art will readily appreciate that method 1800 of FIG. 18 is merely example, and many other methods that include subsets of the steps of this method may be devised in accordance with changes to and/or alternative uses of allergen analysis system 200 of FIG. 2.

It is to be noted, though described using a wearable device 204, in some embodiments, any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

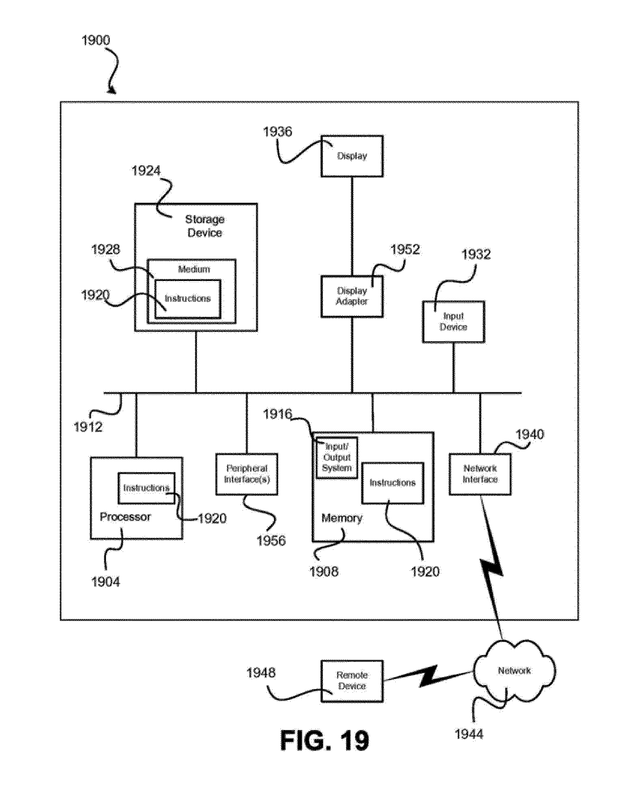

FIG. 19 shows a diagrammatic representation of one embodiment of a computing device in the example form of a computer system 1900 within which a set of instructions for causing a control system, such as any one or more of various systems of the present disclosure, such as the systems illustrated in other figures of this disclosure, as well as systems that would be apparent to those of ordinary skill in the art after reading this entire disclosure, to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1900 includes a processor 1904 and a memory 1908 that communicate with each other, and with other components, via a bus 1912. Bus 1912 may include any of several types of bus structures including, but not limited to, a memory bus, a memory controller, a peripheral bus, a local bus, and any combinations thereof, using any of a variety of bus architectures.

Memory 1908 may include various components (e.g., machine-readable media) including, but not limited to, a random access memory component, a read only component, and any combinations thereof. In one example, a basic input/output system 1916 (BIOS), including basic routines that help to transfer information between elements within computer system 1900, such as during start-up, may be stored in memory 1908. Memory 1908 may also include (e.g., stored on one or more machine-readable media) instructions (e.g., software) 1920 embodying any one or more of the aspects and/or methodologies of the present disclosure. In another example, memory 1908 may further include any number of program modules including, but not limited to, an operating system, one or more application programs, other program modules, program data, and any combinations thereof.

Computer system 1900 may also include a storage device 1924. Examples of a storage device (e.g., storage device 1924) include, but are not limited to, a hard disk drive, a magnetic disk drive, an optical disc drive in combination with an optical medium, a solid-state memory device, and any combinations thereof. Storage device 1924 may be connected to bus 1912 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1924 (or one or more components thereof) may be removably interfaced with computer system 1900 (e.g., via an external port connector (not shown)). Particularly, storage device 1924 and an associated machine-readable medium 1928 may provide nonvolatile and/or volatile storage of machine-readable instructions, data structures, program modules, and/or other data for computer system 1900. In one example, software 1920 may reside, completely or partially, within machine-readable medium 1928. In another example, software 1920 may reside, completely or partially, within processor 1904.

Computer system 1900 may also include an input device 1932. In one example, a user of computer system 1900 may enter commands and/or other information into computer system 1900 via input device 1932. Examples of an input device 1932 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1932 may be interfaced to bus 1912 via any of a variety of interfaces (not shown) including, but not limited to, a serial interface, a parallel interface, a game port, a USB interface, a FIREWIRE interface, a direct interface to bus 1912, and any combinations thereof. Input device 1932 may include a touch screen interface that may be a part of or separate from display 1936, discussed further below. Input device 1932 may be utilized as a user selection device for selecting one or more graphical representations in a graphical interface as described above.

A user may also input commands and/or other information to computer system 1900 via storage device 1924 (e.g., a removable disk drive, a flash drive, etc.) and/or network interface device 1940. A network interface device, such as network interface device 1940, may be utilized for connecting computer system 1900 to one or more of a variety of networks, such as network 1944, and one or more remote devices 1948 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1944, may employ a wired and/or a wireless mode of communication. In general, any network topology may be used. Information (e.g., data, software 1920, etc.) may be communicated to and/or from computer system 1900 via network interface device 1940.

Computer system 1900 may further include a video display adapter 1952 for communicating a displayable image to a display device, such as display device 1936. Examples of a display device include, but are not limited to, a liquid crystal display (LCD), a cathode ray tube (CRT), a plasma display, a light emitting diode (LED) display, and any combinations thereof. Display adapter 1952 and display device 1936 may be utilized in combination with processor 1904 to provide graphical representations of aspects of the present disclosure. In addition to a display device, computer system 1900 may include one or more other peripheral output devices including, but not limited to, an audio speaker, a printer, and any combinations thereof. Such peripheral output devices may be connected to bus 1912 via a peripheral interface 1956. Examples of a peripheral interface include, but are not limited to, a serial port, a USB connection, a FIREWIRE connection, a parallel connection, and any combinations thereof.

In one embodiment, a first independent claim is disclosed and directed to an apparatus, the apparatus comprising: one or more sensors worn by a user; and a processor configured to: receive sensor data, the sensor data corresponding to indicators of an allergic reaction; present questions to the user to solicit answers from the user relating to the allergic reaction based on the sensor data; receive the answers from the user; and automatically provide health information to the user based on the answers, the health information relating to the allergic reaction. The apparatus of the first independent claim, wherein the processor is further configured to, prior to the presenting of the questions: receive an event time that commences a reaction time window as a function of said received sensor data; and determine whether or not the reaction time window has closed, wherein when the determination is that the reaction time window has not closed, initiate the presenting of the questions. The apparatus of the first independent claim, further comprising a user interface, wherein the processor is configured to present the questions, receive the answers, and provide the health information via the user interface. The apparatus of the prior claim, wherein the user interface comprises a display screen. The apparatus of the first independent claim, wherein the processor is further configured to present the health information based on comparing the sensor data with stored condition and symptom data. The apparatus of the first independent claim, wherein the processor is further configured to receive additional sensor data corresponding to non-physiological data, the additional sensor data including any one or a combination of environmental data, data corresponding to an identity of food, or data corresponding to an identity of components of the food, the presenting of the questions based on the additional sensor data. The apparatus of the first independent claim, wherein the processor is further configured to present the questions only if the sensor data indicates that the user is experiencing at least two symptoms corresponding to the allergic reaction. The apparatus of the first independent claim, wherein the processor is further configured to: assign a value to each of the answers received from the user; and generate a total of the values of the answers, wherein the health information is based on the total. The apparatus of the prior claim, wherein the processor is further configured to associate the total of the values of the answer to a health-risk level selected among a plurality of available health risk levels, wherein the processor is further configured to provide an alert corresponding to the health-risk level. In one embodiment, the processor is further configured to present a suggested remedy, provide the health information to a third party device, or both present the suggested remedy and provide the health information to the third party. In one embodiment, the processor is further configured to initiate a remedy user interface and re-present the questions to the user via the remedy user interface. The apparatus of the first independent claim, wherein the health information further comprises an elimination diet and associated coaching. Note that in some embodiments, a single sensor may be used by in the apparatus of the first independent claim. In some embodiments, the indicators may be received from external sensors or devices (e.g., not worn by the user) that communicate the indicators to the apparatus. In some embodiments, the elimination diet may be embodied as an iterative elimination diet that is used with an aim to predict a chance of possible food allergens, including based on accumulated data on the diet prior to allergy outbreaks.

In one embodiment, a second independent claim is disclosed and directed to a machine-readable storage medium containing machine-executable instructions that causes one or more processors of a wearable device comprising plural wearable sensors to: receive sensor data from one or more of the plural wearable sensors, the sensor data corresponding to physiological symptoms corresponding to an allergic reaction; present questions to the user to solicit answers from the user relating to the allergic reaction based on the sensor data; receive the answers from the user; and automatically provide health information to the user based on the answers, the health information relating to the allergic reaction. The machine-readable storage medium of the second independent claim, wherein the machine-executable instructions cause the one or more processors to, prior to the presenting of the questions: receive an event time that commences a reaction time window as a function of said received sensor data; and determine whether or not the reaction time window has closed, wherein when the determination is that the reaction time window has not closed, initiate the presenting of the questions. The machine-readable storage medium of the second independent claim, wherein the machine-executable instructions cause the one or more processors to present the questions, receive the answers, and provide the health information via a user interface, wherein the user interface comprises a display screen. The machine-readable storage medium of the second independent claim, wherein the machine-executable instructions cause the one or more processors to present the health information based on comparing the sensor data with stored condition and symptom data. The machine-readable storage medium of the second independent claim, wherein the machine-executable instructions cause the one or more processors to receive additional sensor data, the additional sensor data including any one or a combination of environmental data, data corresponding to an identity of food, or data corresponding to an identity of components of the food, the presenting of the questions based on the additional sensor data. The machine-readable storage medium of the second independent claim, wherein the machine-executable instructions cause the one or more processors to present the questions only if the sensor data indicates that the user is experiencing at least two symptoms corresponding to the allergic reaction. The machine-readable storage medium of the second independent claim, wherein the machine-executable instructions cause the one or more processors to: assign a value to each of the answers received from the user; generate a total of the values of the answers, wherein the health information is based on the total; associate the total of the values of the answer to a health-risk level selected among a plurality of available health risk levels and provide an alert corresponding to the health-risk level; and present one or any combination of a suggested remedy, provide an elimination diet and associated coaching, or provide the health information to a third party device.

In one embodiment, a third independent claim is disclosed and directed to a method, comprising: receiving sensor data from one or more wearable sensors, the sensor data corresponding to physiological symptoms corresponding to an allergic reaction; presenting questions to a user to solicit answers from the user relating to the allergic reaction based on the sensor data; receiving the answers from the user; and automatically providing health information to the user based on the answers, the health information relating to the allergic reaction.

The foregoing has been a detailed description of illustrative embodiments of the invention. Various modifications and additions can be made without departing from the spirit and scope of this invention. For instance, the wearable device 204 may be worn by a child, and the sensor data may be communicated from the wearable device 204 to other wearable devices 204 and/or other devices 206, 208 to alert, for instance, a parent or guardian when the child is experiencing an allergic reaction and yet may be too young to negotiate the question and answer session. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve various aspects of the present disclosure. Accordingly, this description is meant to be taken only by way of example, and not to otherwise limit the scope of this invention.

Example embodiments have been disclosed above and illustrated in the accompanying drawings. It will be understood by those skilled in the art that various changes, omissions and additions may be made to that which is specifically disclosed herein without departing from the spirit and scope of the present invention.

1. An apparatus, comprising:
one or more sensors worn by a user; and
a processor configured to:
receive indicators of an allergic reaction;
present a first set of questions to the user to solicit answers from the user relating to the allergic reaction based on the indicators;
receive the answers to the first set of questions from the user;
provide a first risk assessment based on the answers;
automatically provide health information to the user based on the answers, the health information relating to the allergic reaction and further comprising a first alert and a first health-risk level, the first health-risk level based on the first risk assessment;
present a second set of questions to the user to solicit answers from the user relating to the allergic reaction based on the indicators;
receive the answers to the second set of questions from the user;
provide a second risk assessment based on the answers; and
automatically provide revised health information to the user based on the answers to the second set of questions, the revised health information relating to the allergic reaction and further comprising a second alert and a second health-risk level, the second health-risk level based on the second risk assessment.

2. The apparatus of claim 1, wherein the processor is further configured to, prior to the presenting of the first set of questions:
receive an event time that commences a reaction time window as a function of said received indicators; and
determine whether or not the reaction time window has closed, wherein when the determination is that the reaction time window has not closed, initiate the presenting of the first set of questions.

3. The apparatus of claim 1, further comprising a user interface, wherein the processor is configured to present the first and second sets of questions, receive the answers to the first and second sets of questions, and provide the health information and the revised health information via the user interface.

4. The apparatus of claim 3, wherein the user interface comprises a display screen.

5. The apparatus of claim 1, wherein the processor is further configured to present the health information based on comparing the indicators with stored condition and symptom data.

6. The apparatus of claim 1, wherein the processor is further configured to receive additional data corresponding to non-physiological data, the additional data including any one or a combination of environmental data, data corresponding to an identity of food, or data corresponding to an identity of components of the food, the presenting of the first set of questions based on the additional data.

7. The apparatus of claim 1, wherein the processor is further configured to present the first set of questions only if the indicators indicate that the user is experiencing at least two symptoms corresponding to the allergic reaction.

8. The apparatus of claim 1, wherein the processor is further configured to:
assign a value to each of the answers received from the user; and
generate a respective total of the values of the answers for the respective first and second sets of questions, wherein the health information and the revised health information are based on the respective total.

9. The apparatus of claim 8, wherein the processor is further configured to associate the respective total of the values of the answers to the respective first and second health-risk levels selected among a plurality of available health-risk levels, wherein the processor is further configured to provide the first and second alerts corresponding to the respective first and second health-risk levels.

10. The apparatus of claim 9, wherein the processor is further configured to present a suggested remedy, provide the health information and the revised health information to a third party device, or both present the suggested remedy and provide the health information and the revised health information to the third party.

11. The apparatus of claim 10, wherein the processor is further configured to initiate a remedy user interface and re-present the first set of questions to the user via the remedy user interface, the re-presented questions comprising the second set of questions.

12. The apparatus of claim 1, wherein the health information further comprises an elimination diet and associated coaching, wherein the processor is further configured to determine foods to avoid based on the indicators and the answers to the first set of questions and determine the health information based on the determination of the foods to avoid.

13. A machine-readable storage medium containing machine-executable instructions that cause one or more processors of a wearable device comprising plural wearable sensors to:
receive sensor data from one or more of the plural wearable sensors, the sensor data corresponding to physiological symptoms corresponding to an allergic reaction;
present a first set of questions to the user to solicit answers from the user relating to the allergic reaction based on the sensor data;
receive the answers to the first set of questions from the user;
provide a first risk assessment based on the answers;
automatically provide health information to the user based on the answers, the health information relating to the allergic reaction and further comprising a first alert and a first health-risk level, the first health-risk level based on the first risk assessment;
present a second set of questions to the user to solicit answers from the user relating to the allergic reaction based on the indicators;
receive the answers to the second set of questions from the user;
provide a second risk assessment based on the answers; and
automatically provide revised health information to the user based on the answers to the second set of questions, the revised health information relating to the allergic reaction and further comprising a second alert and a second health-risk level, the second health-risk level based on the second risk assessment.

14. The machine-readable storage medium of claim 13, wherein the machine-executable instructions cause the one or more processors to, prior to the presenting of the first set of questions:
receive an event time that commences a reaction time window as a function of said received sensor data; and
determine whether or not the reaction time window has closed, wherein when the determination is that the reaction time window has not closed, initiate the presenting of the first set of questions.

15. The machine-readable storage medium of claim 13, wherein the machine-executable instructions cause the one or more processors to present the first and second sets of questions, receive the answers to the first and second sets of questions, and provide the health information and the revised health information via a user interface, wherein the user interface comprises a display screen.

16. The machine-readable storage medium of claim 13, wherein the machine-executable instructions cause the one or more processors to present the health information based on comparing the sensor data with stored condition and symptom data.

17. The machine-readable storage medium of claim 13, wherein the machine-executable instructions cause the one or more processors to receive additional sensor data, the additional sensor data including any one or a combination of environmental data, data corresponding to an identity of food, or data corresponding to an identity of components of the food, the presenting of the first set of questions based on the additional sensor data.

18. The machine-readable storage medium of claim 13, wherein the machine-executable instructions cause the one or more processors to present the first set of questions only if the sensor data indicates that the user is experiencing at least two symptoms corresponding to the allergic reaction.

19. The machine-readable storage medium of claim 13, wherein the machine-executable instructions cause the one or more processors to:
assign a value to each of the answers received from the user;
generate a respective total of the values of the answers for the respective first and second sets of questions, wherein the health information and the revised health information are based on the respective total;
associate the respective total of the values of the answers to the respective first and second health-risk levels selected among a plurality of available health-risk levels and provide the first and second alerts corresponding to the respective first and second health-risk levels; and
present one or any combination of a suggested remedy, provide an elimination diet and associated coaching based on determining foods to avoid according to the answers to the first set of questions and the indicators, or provide the health information and the revised health information to a third party device.

20. A method, comprising:
receiving sensor data from one or more wearable sensors, the sensor data corresponding to physiological symptoms corresponding to an allergic reaction;
presenting a first set of questions to a user to solicit answers from the user relating to the allergic reaction based on the sensor data;
receiving the answers to the first set of questions from the user;
providing a first risk assessment based on the answers;
automatically providing health information to the user based on the answers, the health information relating to the allergic reaction and further comprising a first alert and a first health-risk level, the first health-risk level based on the first risk assessment;
presenting a second set of questions to the user to solicit answers from the user relating to the allergic reaction based on the indicators;
receiving the answers to the second set of questions from the user;
providing a second risk assessment based on the answers; and
automatically providing revised health information to the user based on the answers to the second set of questions, the revised health information relating to the allergic reaction and further comprising a second alert and a second health-risk level, the second health-risk level based on the second risk assessment.
